(19) United States
(12) Patent Application Publication
(10) Pub. No.: US 2009/0163942 A1
(43) Pub. Date: Jun. 25, 2009

(54) Tracheostomy punch dilator

(76) Inventors: Brian J. Cuevas, Cumming, GA (US); Michael Sleva, Atlanta, GA (US); Joe Cesa, Cumming, GA (US); Sam Chan, Alpharetta, GA (US); Nathan Griffith, Rosewell, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 12/004,245

(22) Filed: Dec. 20, 2007

(51) Int. Cl. A61B 17/32; A61M 29/00

(52) U.S. Cl. 606/167; 606/191

(57) ABSTRACT

There is provided a device for performing an initial piercing and dilating of a patient's trachea. The device has a needle within and extending beyond a sheath. After the needle is used to pierce the trachea, it may be removed and a guide wire (J-wire) inserted. A introducer dilator surrounds the sheath and is slidable over the sheath. The introducer dilator has a first position away from the distal end of the sheath and a second position in which the distal end of the sheath is substantially covered by the introducer dilator. The introducer dilator is then used to expand the initial piercing.

### BACKGROUND OF THE INVENTION

Ventilators or respirators are used for mechanical ventilation of the lungs of a patient in a medical setting. The ventilator unit is connected to a hose set; the ventilation tubing or tubing circuit, delivering the ventilation gas to the patient. At the patient end, the ventilation tubing is typically connected to a tracheal ventilation catheter or tube, granting direct and secure access to the lower airways of a patient. Tracheal catheters are equipped with an inflated sealing balloon element, or “cuff”, creating a seal between the tracheal wall and tracheal ventilation tube shaft, permitting positive pressure ventilation of the lungs.

One type of tracheal catheter, an endotracheal tube (ET tube), inserted through the mouth, is generally used for a number of days before a decision is made to switch a patient to a tracheostomy tube, inserted directly into the trachea through an ostomy in the tracheal wall. Endotracheal tubes have been linked in some studies to an increased rate of ventilator acquired pneumonia (VAP) and so tracheostomy operations are becoming increasingly common and are being performed earlier in the patient's hospital stay in order to reduce the occurrence of VAP.

A tracheostomy procedure involves making an incision in the skin of the neck to grant access to the trachea. Because of the uniquely flexible and elastic nature of the trachea, it has been found that healing is much faster if only a small hole is made in the trachea and the hole dilated, rather than cutting the trachea. After the skin incision, a hemostat or other implement may be used to separate the subcutaneous tissues to gain access to the trachea, and digital palpation is used to locate the tracheal rings. A bronchoscope is usually inserted into the ET tube and the tube withdrawn from the trachea until the light of the bronchoscope transdermally illuminates the site of the incision. A sheathed needle is used to puncture the trachea from the outside, usually between the second and third tracheal rings, the needle is removed with the sheath remaining, a flexible guide wire (also called a J-wire) is inserted in the place of the needle and then the sheath is removed. The bronchoscope is used for viewing the procedure from inside the trachea in order to avoid damage to the rear tracheal wall. A small (e.g. 14 French) introducer dilator is introduced over the guide wire to perform an initial dilation of the trachea and the dilator is then removed. A smaller (e.g. 8 French) guiding catheter is then introduced over the guide wire. (Note, French is a measure of circumference based on the theory that non-round tubes of the same circumference will fit into the same incision. One French is approximately 0.33 mm or 0.013 inch).

After the guiding catheter is introduced, a first dilator such as the Cook Medical Inc. Blue Rhino® dilator (see also U.S. Pat. No. 6,637,435), is placed over the guide wire and the guiding catheter and first dilator are advanced into the trachea as a unit to perform the dilation. Cook Medical recommends a slight over-dilation in order to make the placement of the tracheostomy tube easier. After dilation, the first dilator is removed and the tracheostomy tube (with cannula removed) is introduced over the guide catheter using a second dilator that fits just inside the trachostomy tube and protrudes about 2 cm beyond the distal end of the tracheostomy tube. The guide catheter, second dilator and tracheostomy tube are advanced into the trachea as a unit. Once the tracheostomy tube is at the proper depth, the second dilator, guide catheter and guide wire are removed through the tracheostomy tube, the inner cannula is inserted into the tracheostomy tube and the tube connected to the ventilator. The procedure is complete.

As can be understood from the above description, the current state of the art for tracheostomy involves numerous steps and the insertion and removal of a number of components before the successful completion of the procedure. For most of this time, the patient is disconnected from the ventilator and is therefore, not breathing. In addition, the large number of parts used in current tracheostomy kits increases the likelihood that an item may be accidentally rendered unsterile and be unable to be used. In such cases, the patient must be re-intubated with an ET tube. Even if the procedure proceeds uneventfully, however, the amount of time the patient is not breathing is significant; on the order of 7 minutes or more. This is clearly a significant event, especially for a patient who is, most likely, not in optimal physical condition.

There remains a need for a device that can more quickly and safely allow for the successful placement of a tracheostomy tube.

### SUMMARY OF THE INVENTION

There is provided a novel punch dilator device that allows for the rapid puncture of the tracheal wall, insertion of a guide wire and initial dilation. The device has a needle within and extending beyond a sheath. After the needle is used to pierce the trachea, it may be removed and a guide wire (J-wire) inserted. An introducer dilator surrounds the sheath and is slidable over the sheath. The introducer dilator has a first position away from the distal end of the sheath and a second position in which the distal end of the sheath is substantially covered by the introducer dilator. The introducer dilator is then used to expand the initial piercing.

### BRIEF DESCRIPTION OF THE DRAWINGS

FIG. 1 illustrates the novel punch dilator with the introducer dilator in the proximal (fully retracted) position.

FIG. 2 illustrates the novel punch dilator with the introducer dilator in the distal (fully extended) position.

FIG. 3 illustrates the separate components of one embodiment of the punch dilator.

### DETAILED DESCRIPTION OF THE INVENTION

Tracheostomy is a lifesaving procedure to allow a patient to be ventilated directly through the trachea. Tracheostomy is also believed by many to prevent or retard the onset of ventilator acquired pneumonia (VAP). This lifesaving procedure is, unfortunately, relatively time consuming and current technology requires a large number of steps and pieces of equipment that must remain sterile and functioning properly in order to arrive at a successful conclusion. This procedure may be greatly improved using the device described in the Summary above; the novel punch dilator (the device). In addition, the device may be used in emergency tracheotomies, and the term “tracheostomy” as used herein is meant to include the term tracheotomy.

The device replaces a number of pieces used in the current state of the art procedure described in the introduction. The device replaces the separate needle, sheath and the introducer dilator and allows for the aspiration of the patient to ensure the needle has entered the trachea and not the esophagus or other tissue. The device is designed so that the procedure is, except of course for the initial piercing of the trachea, completely reversible at any point during the procedure. The locking mechanism used to control the sliding introducer dilator in first and second positions allows great flexibility in reversing the procedure if necessary. Likewise, the body of the device allows for the reintroduction of the needle after it is removed, should that become necessary.

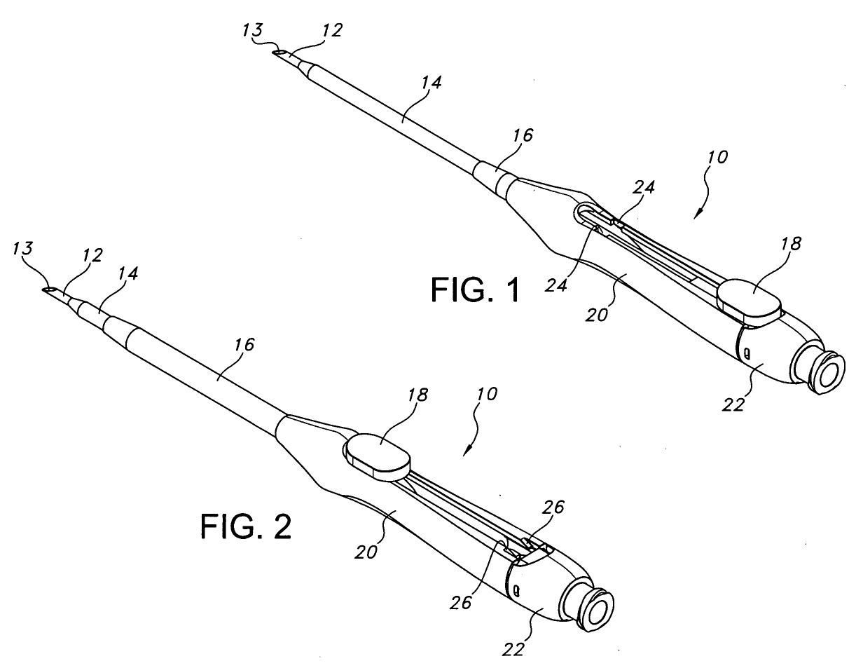

Turning to FIG. 1, one embodiment of the device 10 has a needle 12 preferably having a beveled distal end 13 for piercing the tracheal wall, a sheath 14 surrounding the needle 12 except for a few millimeters on the extreme distal end, an introducer dilator 16 portion over the sheath 14 which may move over the sheath 14, and a slidable and lockable button 18 to control the movement of the introducer dilator 16 over the sheath 14 within the body 20 of the device 10. The sheath 14 and dilator 16 may have tapered distal ends. The needle 12 extends for the length of the device 10 and is in communication with an aspirating connection 22 on the proximal end 15 of the needle 12 for connection to an aspirating syringe to ensure that the needle 12 has been placed in the trachea.

In this embodiment, before use, the slidable button 18 is moved into the most proximal position, the sheath 14 is exposed and the introducer dilator 16 is retracted (FIG. 1). It is in this first position that the device 10 is used to pierce the tracheal wall. The slidable button 18 may be releasably locked in either the proximal or distal position by a locking mechanism using slots 24 (FIG. 1) and tabs 26 (FIG. 2). The button 18 is functionally connected to the introducer dilator 16 such that they move as one unit. In this embodiment, when the button 18 is moved toward the distal end, projections on the bottom of the button 18 mate with the slots 24 on the body 20 to hold the button 18 and therefore the introducer dilator 16 in position. When the button 18 is moved toward the proximal end, members on the bottom of the button 18 mate with the tabs 26 on the body 20 to hold the button 18 and therefore the introducer dilator 16 in position. The application of moderate force by the fingers to the button 18 in either position will move the button 18 out of position without breaking any component.

Once the needle 12 is inserted into the trachea, a syringe (not shown), may be used to aspirate the patient through an inner cannula of the needle 12, the needle 12 extending through the sheath 14, sheath holder 19 and body 20 of the device 10 and being operatively connected to the aspirating (proximal) connection 22. Once it has been determined that the needle 12 has indeed entered the trachea, the needle 12 may be then withdrawn from the trachea as well as from the device 10, with the sheath 14 remaining in the trachea. A J-wire may then be inserted in place of the needle 12 through the device 10 and into the trachea.

After the J-wire is introduced, the device 10 may be retracted slightly from the patient's trachea to allow for the introducer dilator 16 to be distally extended without obstruction. The introducer dilator 16 is then moved toward the distal end of the device 10 by the action of the slidable button 18 on the body 20 (FIG. 2). Once the introducer dilator 16 is extended distally in the second position and locked in place using the button 18 and slots 24 to prevent accidental retraction of introducer dilator 16, the introducer dilator 16 may be advanced into the trachea to expand the initial piercing created by the needle 12. The entire device 10 may then be removed, leaving only the J-wire in place for the completion of the tracheostomy procedure.

At any time during the procedure, the steps outlined above may be reversed and the device 10 removed from the trachea. The button 18 may be unlocked to retract the introducer dilator 16, for example, or the needle 12 may be reinserted through the body 20 of the device 10 (provided the J-wire has not been inserted or has been removed). The entire device 10 may be removed at any point in the procedure should the need arise. This allows great flexibility and control for the health care professional should there be an unforeseen complication that requires the reversal or immediate cessation of the procedure.

As can be seen in FIG. 1 the needle 12 is within and extending beyond the distal end of the sheath 14. The introducer dilator 16 circumferentially surrounds the sheath 14 and is slidable over the sheath 14 in the lengthwise direction. The sheath 14 is stationary with respect to the needle 12 and the body 20 of the device 10. The introducer dilator 16 has a first position away from the distal end of the sheath 14 and substantially uncovering the sheath 14, e.g. the fully retracted or proximal position (FIG. 1) and a second position in which the distal end of the sheath 14 is substantially covered by the introducer dilator 16, e.g. the fully extended or distal position (FIG. 2). The button 18 may be used to lock the introducer dilator 16 in either the distal or proximal position.

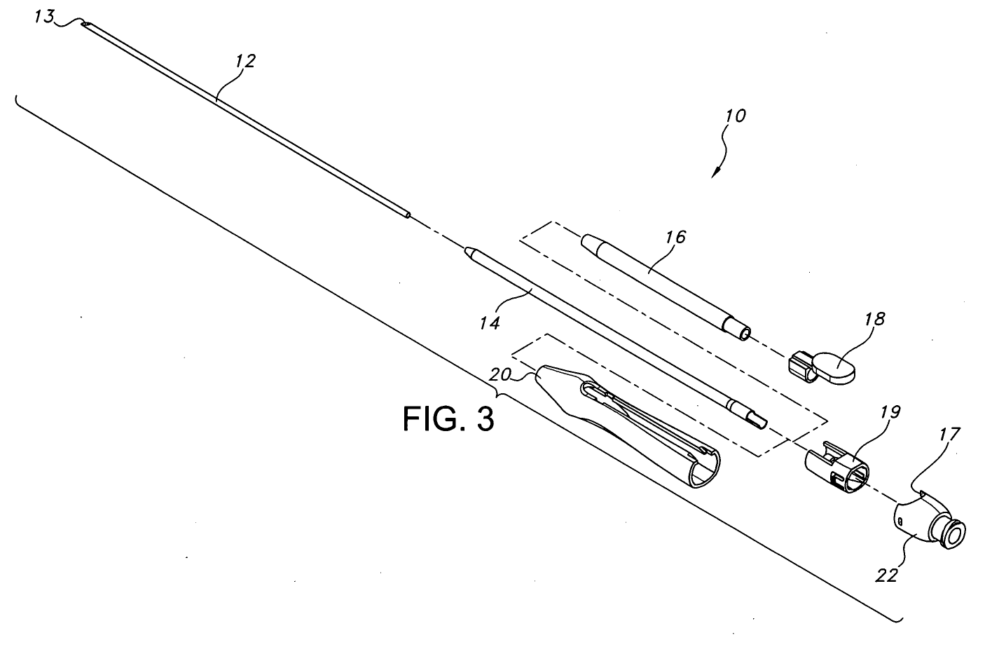

For ease of manufacture the components of the device 10 may be made as a number of separate parts and assembled to produce the final device 10. Turning to FIG. 3 one can see illustrated the parts of the device 10 as separate components. This is meant only as one means of or suggestion for producing the device and is not meant as a limitation or restriction of the inventive concept. In this embodiment there are three pre-assemblies in the device 10; the dilator 16 and button 18 pre-assembly, the sheath 14, sheath holder 19 and body 20 pre-assembly, and the needle and aspirating connection 22 pre-assembly. Assembly of the parts of FIG. 3 would involve attaching the button 18 to the dilator 16, inserting the sheath 14 through the dilator/button assembly 16/18 with the button 18 nearer the proximal end of the sheath 14, permanently connecting the proximal end of the sheath 14 with the sheath holder 19 and inserting the entire assembly into the body 20. In this embodiment the sheath holder 19 slips into the proximal end of the body 20 and locks into position. Once assembled, the sheath 14, sheath holder 19 and body 20 cannot move relative to each other. Likewise the dilator 16 and button 18 cannot move relative to each other after assembly. The needle 12 and aspirating connection 22 may be joined separately and inserted into the sheath 14 through the sheath holder 19.

The aspirating connection 22 on the proximal end 15 of the needle 12 has a releasable locking mechanism 17 to hold the needle 12 in place on the sheath holder 19 in order to prevent a change in the position of the needle 12 relative to the body 20. The distal end of the needle 12 has a bevel 13 and the locking mechanism 17 helps maintain the bevel orientation as well as helping to prevent the accidental separation of the needle 12 from the body 20. The locking mechanism 17 may be a “push and twist” lock mechanism having a keyed entrance so that the needle may be inserted correctly in only one position, similar to that found on “child proof” medication bottles. When the needle is inserted, for example, a tab on the proximal end of the needle 12 may align with a slot in the body 10 and, once inserted, the needle 12 may be turned slightly to its final position. Alternatively the locking mechanism 17 may be a tab and slot arrangement as shown in FIG. 3 wherein the aspirating connection 22 releasably locks onto both sides of the sheath holder 19. Other means of locking the needle 12 into the body 20 are known to those skilled in the art and may be used, e.g. close tolerances that lock with friction, a ball point pen type lock, a small cotter pin arrangement, etc.

After the initial piercing of the trachea by the needle 12, the cannula of the needle 12 may be used to aspirate a patient with a syringe through the aspirating connection 22 to ensure that the needle has entered the trachea. With the sheath 14 in the trachea, the needle 12 may then be removed and a J-wire inserted though the sheath 14 into the trachea in place of the needle 12. The introducer dilator 16 may be moved from the first to second positions by sliding it out of the body 20 using a finger activated button 18 or by other means, where it desirably locks in position. Instead of the means for moving the dilator as described in the embodiment above, other means known in the art may be used, like, for example, a conventional twisting mechanism wherein the introducer dilator 16 moves as a result of the proximal end of the device 10 being twisted, a ball point pen spring type mechanism, or an electrically controlled motor may be used to move the dilator 16,. The introducer dilator 16 may then be used to expand the initial piercing of the trachea by the needle 12 and may then be unlocked and withdrawn. The procedure may then continue in the conventional manner discussed in the introduction, i.e. from the point of inserting the guiding catheter on the J-wire.

While the exact size of the punch dilator device may be varied, there are some recommended criteria that should be met. The device, for example, should have a total length of less than 25 cm, more particularly less than 18 cm and weigh less that about 20 grams, more particularly less than 10 gms. The device must be biocompatible, free of di(2-ethylhexyl)phthalate (DEHP) and preferably free of animal derived products. The needle may be from 1 to 15 French, more particularly between 2 and 8 French and desirably about 4.5 French in size and the longest component of the device. The sheath is slightly larger than the needle; about 1 to 15 French, more particularly about 2 to 8 French, desirably about 6 French in size. The introducer dilator is from 5 to 20 French, more particularly between 11 and 18 French and desirably about 14 French and can be from about 40 to 70 mm long, more particularly between 45 and 65 mm and desirably about 50 to 55 mm long. When the introducer dilator is fully extended, from 1 to 20 mm more particularly between 3 and 10 mm and desirably about 6 mm of the sheath and from 1 to 30 mm, more particularly between 2 and 10 mm and desirably about 4 mm of the distal end of the needle beyond (distal to) the sheath should be visible. When the introducer dilator is fully retracted about 20 to 80 mm, more particularly between 30 and 55 mm and desirably 40 to 45 mm of the sheath and about 1 to 30 mm, more particularly between 2 and 10 mm and desirably 4 mm of the distal end of the needle should be visible. The difference, therefore, in the amount of introducer dilator visible outside the body 20, between the introducer dilator fully retracted and extended positions, is between about 20 and 100 mm, more particularly between 25 and 55 mm and desirably about 35 and 40 mm, linearly along the sheath. It is recommended that the force required to separate the components of the device be not greater than 30 Newtons. The guide wire should be about 0.052 inches (0.020 cm) in diameter and pass through the device using a force of no more than 2 Newtons.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A tracheostomy punch dilator comprising a needle within and extending beyond a distal end of a sheath, having an introducer dilator surrounding said sheath and slidable over said sheath, wherein said introducer dilator has a first position away from the distal end of said sheath and a second position in which said distal end of said sheath is substantially covered by said introducer dilator.

2. The dilator of claim 1 wherein said introducer dilator is housed within a body when in said first position.

3. The dilator of claim 1 wherein said needle has a cannula.

4. The dilator of claim 3 wherein said cannula of said needle may be used to aspirate a patient after said needle is used to pierce a trachea.

5. The dilator of claim 4 wherein said needle is removable from said body.

6. The dilator of claim 1 wherein said dilator comprises a means of moving said introducer dilator between said first and second positions.

7. The dilator of claim 6 wherein said means comprises sliding said introducer dilator between said first and second positions.

8. The dilator of claim 6 wherein said means comprises twisting said dilator to move said dilator between said first and second positions.

9. The dilator of claim 6 wherein said introducer dilator releasably locks into said first and second positions.

10. The dilator of claim 1 wherein said introducer dilator is between 5 to 20 French.

11. The dilator of claim 1 wherein said introducer dilator is 14 French.

12. The dilator of claim 1 wherein said needle is between 1 and 15 French.

13. A punch dilator for performing a tracheostomy comprising a body enclosing a 11 to 18 French introducer dilator that is between 45 and 65 mm long and that is movable from a first position to a second position, said introducer dilator surrounding a stationary sheath that extends beyond the distal end of said introducer dilator and in which is located a removable 2 to 8 French needle having a cannula, wherein said needle extends beyond a distal end of said sheath, wherein said introducer dilator is, in said first position, substantially uncovering said distal end of said sheath and, in said second position, substantially covering said distal end of said sheath.

14. The dilator of claim 13 wherein introducer dilator is slidably movable from said first to said second position.

15. The dilator of claim 14 wherein said introducer dilator releasably locks into said first and second positions.

16. The dilator of claim 13 wherein said cannula may be used to aspirate a patient.

17. The dilator of claim 13 wherein said needle is used to pierce a trachea and said needle is removed from said dilator.

18. The dilator of claim 17 wherein said needle may be reinstalled in said dilator.

19. The dilator of claim 17 wherein said introducer dilator is moved and releasably locked into said second position and used to dilate said trachea.

20. The dilator of claim 19 wherein said introducer dilator is moved and releasably locked into said first position after dilation of said trachea.
